(19) United States
(12) Patent Application Publication
(10) Pub. No.: US 2012/0258052 A1
(43) Pub. Date: Oct. 11, 2012

(54) AEROSOL COMPRISING A STARCH COMPOUND AND A (C8 TO C20) MONOCARBOXYLIC ACID COMPOUND

(76) Inventors: Burkhard Mueller, Hamburg (DE); Ulrike Heinsohu, Hamburg (DE)

(21) Appl. No.: 13/526,069

(22) Filed: Jun. 18, 2012

Related U.S. Application Data
(63) Continuation of application No. PCT/EP2010/067624, filed on Nov. 17, 2010.

(30) Foreign Application Priority Data
Dec. 18, 2009 (DE) 102009054978.1

(51) Int. Cl. A61K 8/73; B65D 83/14; A61K 8/02

(52) U.S. Cl. 424/47; 222/635

(57) ABSTRACT

Cosmetic products for treating keratin fibers, especially human hair, containing, in a cosmetic carrier, at least one starch compound, at least 0.01% by weight of a (C₈ to C₂₀) monocarboxylic acid compound of formula (I),

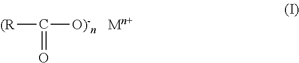

wherein R is a (C₇ to C₁₉) alkyl group, Mⁿ⁺ is a cation of n valency and n is 1, 2 or 3 (especially 2), and at least one blowing agent, wherein the products can be dispensed from an aerosol container without the valve excessively clogging up or being blocked.

### CROSS REFERENCE TO RELATED APPLICATIONS

The present application is a continuation of International Application No. PCT/EP2010/067624 filed 17 Nov. 2010, which claims priority to German Patent Application No. 10 2009 054 978.1, filed 18 Dec. 2009, both of which are incorporated herein by reference.

### BACKGROUND OF THE INVENTION

The present invention relates to cosmetic agents comprising in a cosmetic carrier at least one starch compound, at least 0.01 wt. % of a (C₈ to C₂₀) monocarboxylic acid compound, and at least one propellant. The invention also provides aerosol dispensing containers comprising at least one valve, at least one nozzle, and at least one storage container containing a cosmetic agent comprising in a cosmetic carrier at least one starch compound, at least 0.01 wt. % of a (C₈ to C₂₀) monocarboxylic acid compound and at least one propellant. Furthermore, a method of treating hair is described in which the aerosols are used.

A cosmetic application of aerosols is known by the consumer, particularly for hair treatment agents, shaving foams or deodorants. In the sector of temporary hair reshaping as well as in the sector of hair cleaning, aerosols are used as foam and particularly as a spray.

Suitable hair cosmetics for temporary shaping contain natural or synthetic polymers as a shaping component. Preparations containing a dissolved or dispersed polymer can be applied onto the hair in finely divided form as an aerosol using propellant gases. Shampoos present in the form of an aerosol include in particular starch-containing dry shampoos mentioned in British Patent No. GB 1235908 for degreasing the hair.

It has been shown that starch-containing cosmetic agents tend to clog or even block both the spray or foam nozzle and the valve of the aerosol container. The present invention therefore provides starch-containing cosmetic agents in aerosol form which do not clog or block the nozzle or valve of an aerosol container.

The invention therefore firstly provides a cosmetic agent for treating keratinic fibers, particularly human hair, comprising in a cosmetic carrier—
at least one starch compound,
at least 0.01 wt. % of a (C₈ to C₂₀) monocarboxylic acid compound of formula (I)

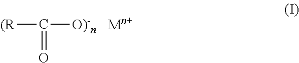

wherein R is a (C₇ to C₁₉) alkyl group, Mⁿ⁺ is an n-valent cation, and n is the number 1, 2 or 3 (particularly 2), and
at least one propellant.

Keratinic fibers according to the invention include furs, wool, feathers and particularly human hair.

Agents according to the invention contain the above-mentioned components and optionally other active substances in a cosmetic carrier. Cosmetic carriers are known to be physiologically acceptable carriers suitable for application on living beings, particularly humans. Within the meaning of the invention, cosmetic carriers are preferably liquid. A cosmetic carrier is regarded as “liquid” if it is liquid at 20° C. and a pressure of 1013.25 mbar.

Preferred cosmetic carriers are alcoholic cosmetic carriers or aqueous-alcoholic cosmetic carriers, particularly liquid alcoholic cosmetic carriers or liquid aqueous-alcoholic cosmetic carriers.

In a preferred embodiment of the agent according to the invention, a liquid alcoholic cosmetic carrier is present (particularly a liquid alcoholic carrier comprising at least one alcohol having 2 to 6 carbon atoms and 1 to 3 hydroxyl groups). It is most particularly preferred if the agent contains a liquid alcoholic carrier and is anhydrous. The term “anhydrous” means that the agents according to the invention contain 0 to no more than 5 wt. %, preferably 0 to no more than 3 wt. %, more preferably 0 to no more than 2 wt. %, of free water, based on total agent.

The alcohol having 2 to 6 carbon atoms and 1 to 3 hydroxyl groups is preferably chosen from at least one compound of ethanol, ethylene glycol, isopropanol, 1,2-propylene glycol, 1,3-propylene glycol, glycerol, n-butanol and 1,3-butylene glycol. A most particularly preferred alcohol is ethanol.

The additional alcohol having 2 to 6 carbon atoms and 1 to 3 hydroxyl groups is present in the agent as a cosmetic carrier, if an alcoholic liquid carrier is present, preferably in an amount of 1 wt. % to 30 wt. %, particularly 3 wt. % to 20 wt. %, based on weight of the cosmetic agent.

The agent according to the invention contains at least one starch compound.

Starch is a reserve carbohydrate which is stored by many plants in the form of grains of starch (granules) generally 1 to 200 μm in size in various parts of plants (e.g., in tubers or roots, cereal seeds, fruits and in the pith). Starch belongs to the family of homoglycans and is a polycondensation product of D-glucose. Starch consists of three structurally different polymers of d-glucopyranose: amylase, amylopectin and an intermediate fraction. Higher plants contain 0 to 45 wt. % amylose, based on dry matter. The intermediate fraction, also referred to as anomalous amylopectin, lies between amylose and amylopectin in terms of structure. Quantitative data for amylopectin defined in the context of this application include the intermediate fraction. Amylose consists of predominantly linear α-1,4-glycosidically linked d-glucose, Mᵣ 50000-150000. The resulting chains form double helices in the starch. As well as the α-1,4 links described for amylose, amylopectin also contains α-1,6 bonds as branching sites in an amount of 4 to 6%. The average spacing between the branching sites is about 12 to 17 glucose units. The molecular weight of 10⁷ to 7·10⁸ corresponds to about 10⁵ glucose units, making amylopectin one of the largest biopolymers. These branchings are distributed along the molecule in such a way that a cluster structure develops with relatively short side chains. Pairs of these side chains form a double helix. Because of the large number of branching sites, amylopectin is relatively highly soluble in water. A starch compound according to the present invention is defined as starch and a polycondensation product of D-glucose obtained from starch or a derivative thereof which is modified with other chemical groups at the OH groups of the polycondensation product of D-glucose. Chemical compounds such as propylene oxide, ethylene oxide, 2-octylsuccinic acid (CAS No.: 2530-32-7) are preferably suitable for modifying these OH groups.

An example of a preferred modified starch compound is aluminum starch octenyl succinate, an aluminum salt of corn starch modified with 2-octylsuccinic acid, obtainable, for example, with the trade name DryFlo Plus or DryFlo Pure from National Starch.

A propylene oxide-modified starch having an average molecular weight (weight average) of 50 to 2500 kDa is another example of a modified starch compound. The corresponding propylene oxide-modified starches are prepared, for example, by reaction of a natural starch with propylene oxide.

The starch component of the agent according to the invention is preferably present as solid particles dispersed in the cosmetic carrier (particularly in a liquid cosmetic carrier). “Solid particles” are solids that are particulate at 20° C. and 1013.25 mbar.

A starch compound that can preferably be used according to the invention is chosen from at least one—optionally modified—polycondensation product of D-glucose obtained from starch from potatoes, corn, rice, peas, acorns, chestnuts, barley, wheat, bananas, sago, millet, sorghum, oats, barley, rye, beans, sweet potato, maranta or cassava. Preferably, the agent according to the invention contains at least one starch compound which is tapioca starch, potato starch, corn starch or rice starch or is derived therefrom. Mixtures of these starch compounds are also included according to the invention. The starch compound is most preferably rice starch.

The starch compound is present in the agent preferably in an amount of 2 to 20 wt. %, particularly 3 to 15.0 wt. %, based on total weight of the agent.

Furthermore, the agent according to the invention contains at least 0.01 wt. % of at least one (C₈ to C₂₀) monocarboxylic acid compound of formula (I) above. Compounds of this type wherein, according to formula (I), n is the number 2 are preferably suitable according to the invention. The effect according to the invention is particularly marked if, according to formula (I), n is 2 and a liquid alcoholic cosmetic carrier is additionally present.

The group Mⁿ⁺ of formula (I) generally is an n-valent cation. For reasons of electroneutrality, this compensates for the single negative charge of the carboxyl group according to formula (I). In principle, all mono-, di- or trivalent cations are suitable as n-valent cations Mⁿ⁺ (n=1, 2 or 3). In particular, Mⁿ⁺ according to formula (I) is preferably chosen from metal cations of the physiologically acceptable metals from groups Ia, Ib, IIa, IIb, IIIb, VIa or VIII of the periodic table, ammonium ions and cationic organic compounds with a quaternized nitrogen atom. The latter are formed, for example, by protonation of primary, secondary or tertiary organic amines with an acid, such as with compounds of formula (I) in their acidic form (Mⁿ⁺=H⁺), or by permanent quaternization of these organic amines. Examples of these cationic organic ammonium compounds are 2-ammonioethanol and 2-trimethylammonioethanol. Mⁿ⁺ according to formula (I) preferably is an ammonium ion (n=1), an alkali metal ion (n=1) (particularly K⁺), an alkaline earth metal ion (n=2) (particularly Ca²⁺, Mg²⁺) or a zinc ion (n=2, i.e., Zn²⁺), more preferably a magnesium ion (Mg²⁺) or a zinc ion (Zn²⁺).

R according to formula (I) is preferably a linear or branched (C₇ to C₂₀) alkyl group, particularly n-heptyl, n-nonyl, n-undecyl, n-tridecyl, n-pentadecyl or n-heptadecyl.

Most preferably, at least one (C₈ to C₂₀) monocarboxylic acid compound of formula (I) is chosen from ammonium stearate, calcium distearate, magnesium distearate, zinc distearate, ammonium isostearate, calcium diisostearate, magnesium diisostearate, zinc diisostearate, ammonium palmitate, calcium dipalmitate, magnesium dipalmitate, zinc dipalmitate or mixtures thereof, with magnesium distearate, zinc distearate or mixtures thereof being most particularly preferably suitable.

The (C₈ to C₂₀) monocarboxylic acid compounds of formula (I) according to the invention are present in the agent preferably in an amount of 0.01 to 5.0 wt. %, more preferably 0.05 to 1.0 wt. %, based on total weight of the agent according to the invention.

It is further preferred to use the starch compound and the (C₈ to C₂₀) monocarboxylic acid compounds of formula (I) in a weight ratio of 40:1 to 5:1, in particular 30:1 to 10:1, in the agent according to the invention.

Agents according to the invention also contain at least one propellant as a component. One possible way of producing the according to the invention is to fill a suitable pressure-resistant container with all the components of the agent with the exception of the propellant, then sealing the container with a valve, and adding the desired quantity of propellant using conventional techniques.

To prepare an agent according to the invention, propellants are preferably chosen from N₂O, dimethyl ether, CO₂, air, alkanes with 3 to 5 carbon atoms, such as propane, n-butane, isobutane, n-pentane and iso-pentane, and mixtures thereof. Dimethyl ether, propane, n-butane, isobutane and mixtures thereof are preferred.

Agents according to the invention contain propellant preferably in amounts of 45 to 90 wt. %, based on total product. Quantities of 50 to 85 wt. %, particularly 60 to 80 wt. %, are particularly preferred.

Most particularly preferred agents according to the invention are cosmetic agents of embodiments (A) to (L):

### (A):

A cosmetic agent for treating keratinic fibers, particularly human hair, comprising in a liquid cosmetic carrier—
at least one starch compound dispersed as solid particles,
at least 0.01 wt. % of a (C₈ to C₂₀) monocarboxylic acid compound of formula (I)

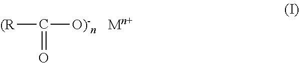

wherein R is a (C₇ to C₁₉) alkyl group, Mⁿ⁺ is a divalent cation, and n is the number 2, and
at least one propellant.

### (B):

A cosmetic agent for treating keratinic fibers, particularly human hair, comprising in a liquid cosmetic carrier—
at least one at least one starch compound dispersed as solid particles, selected from at least one—optionally modified—polycondensation product of D-glucose obtained from starch from potatoes, corn, rice, peas, acorns, chestnuts, barley, wheat, bananas, sago, millet, sorghum, oats, barley, rye, beans, sweet potato, maranta or cassava,
at least 0.01 wt. % of a (C₈ to C₂₀) monocarboxylic acid compound of formula (I)

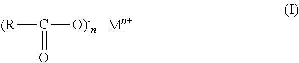

wherein R is a (C₇ to C₁₉) alkyl group, Mⁿ⁺ is a divalent cation, and n is the number 2, and
at least one propellant.

### (C):

A cosmetic agent for treating keratinic fibers, particularly human hair, comprising in a liquid cosmetic carrier—
at least one starch compound dispersed as solid particles, selected from at least one—optionally modified—polycondensation product of D-glucose obtained from starch from potatoes, corn, rice, peas, acorns, chestnuts, barley, wheat, bananas, sago, millet, sorghum, oats, barley, rye, beans, sweet potato, maranta or cassava,
at least 0.01 wt. % of a (C₈ to C₂₀) monocarboxylic acid compound selected from ammonium stearate, calcium distearate, magnesium distearate, zinc distearate, ammonium isostearate, calcium diisostearate, magnesium diisostearate, zinc diisostearate, and
at least one propellant.

### (D):

A cosmetic agent for treating keratinic fibers, particularly human hair, comprising in a liquid alcoholic cosmetic carrier containing at least one alcohol having 2 to 6 carbon atoms and 1 to 3 hydroxyl groups—
at least one starch compound dispersed as solid particles,
at least 0.01 wt. % of a (C₈ to C₂₀) monocarboxylic acid compound of formula (I)

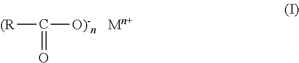

wherein R is a (C₇ to C₁₉) alkyl group, Mⁿ⁺ is a divalent cation, and n is the number 2, and
at least one propellant.

### (E):

A cosmetic agent for treating keratinic fibers, particularly human hair, comprising in a liquid alcoholic cosmetic carrier containing at least one alcohol having 2 to 6 carbon atoms and 1 to 3 hydroxyl groups—
at least one at least one starch compound dispersed as solid particles, derived from at least one starch from potatoes, corn, rice, peas, acorns, chestnuts, barley, wheat, bananas, sago, millet, sorghum, oats, barley, rye, beans, sweet potato, maranta or cassava.
at least 0.01 wt. % of a (C₈ to C₂₀) monocarboxylic acid compound of formula (I)

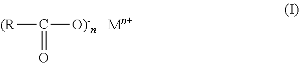

wherein R is a (C₇ to C₁₉) alkyl group, Nⁿ⁺ is a divalent cation, and n is the number 2, and
at least one propellant.

### (F):

A cosmetic agent for treating keratinic fibers, particularly human hair, comprising in a liquid alcoholic cosmetic carrier containing at least one alcohol having 2 to 6 carbon atoms and 1 to 3 hydroxyl groups—
at least one starch compound dispersed as solid particles, chosen from at least one—optionally modified—polycondensation product of D-glucose obtained from starch from potatoes, corn, rice, peas, acorns, chestnuts, barley, wheat, bananas, sago, millet, sorghum, oats, barley, rye, beans, sweet potato, maranta or cassava,
at least 0.01 wt. % of a (C₈ to C₂₀) monocarboxylic acid compound chosen from ammonium stearate, calcium distearate, magnesium distearate, zinc distearate, ammonium isostearate, calcium diisostearate, magnesium diisostearate, zinc diisostearate, and
at least one propellant.

### (G):

A cosmetic agent for treating keratinic fibers, particularly human hair, comprising in a liquid cosmetic carrier—
2 to 20 wt. %, particularly 3 to 15.0 wt. %, of at least one starch compound dispersed as solid particles,
0.01 to 5.0 wt. %, particularly 0.05 to 1.0 wt. %, of at least one (C₈ to C₂₀) monocarboxylic acid compound of formula (I)

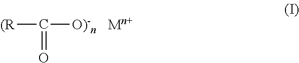

wherein R is a (C₇ to C₁₉) alkyl group, Mⁿ⁺ is a divalent cation, and n is the number 2, and
at least one propellant.

### (H):

A cosmetic agent for treating keratinic fibers, particularly human hair, comprising in a liquid cosmetic carrier—
2 to 20 wt. %, particularly 3 to 15.0 wt. %, of at least one at least one starch compound dispersed as solid particles, chosen from at least one—optionally modified—polycondensation product of D-glucose obtained from starch from potatoes, corn, rice, peas, acorns, chestnuts, barley, wheat, bananas, sago, millet, sorghum, oats, barley, rye, beans, sweet potato, maranta or cassava,
0.01 to 5.0 wt. %, particularly 0.05 to 1.0 wt. %, of at least one (C₈ to C₂₀) monocarboxylic acid compound of formula (I)

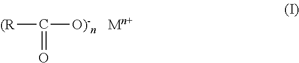

wherein R is a (C₇ to C₁₉) alkyl group, Mⁿ⁺ is a divalent cation, and n is the number 2, and
at least one propellant.

A cosmetic agent for treating keratinic fibers, particularly human hair, comprising in a liquid cosmetic carrier—
2 to 20 wt. %, particularly 3 to 15.0 wt. %, of at least one starch compound dispersed as solid particles, chosen from at least one—optionally modified—polycondensation product of D-glucose obtained from starch from potatoes, corn, rice, peas, acorns, chestnuts, barley, wheat, bananas, sago, millet, sorghum, oats, barley, rye, beans, sweet potato, maranta or cassava,
0.01 to 5.0 wt. %, particularly 0.05 to 1.0 wt. %, of at least one (C₈ to C₂₀) monocarboxylic acid compound chosen from ammonium stearate, calcium distearate, magnesium distearate, zinc distearate, ammonium isostearate, calcium diisostearate, magnesium diisostearate, zinc diisostearate, and
at least one propellant.

### (J):

A cosmetic agent for treating keratinic fibers, particularly human hair, comprising in a liquid alcoholic cosmetic carrier containing at least one alcohol having 2 to 6 carbon atoms and 1 to 3 hydroxyl groups—
2 to 20 wt. %, particularly 3 to 15.0 wt. %, of at least one starch compound dispersed as solid particles,
0.01 to 5.0 wt. %, particularly 0.05 to 1.0 wt. %, of at least one (C_{B} to C₂₀) monocarboxylic acid compound of formula (I)

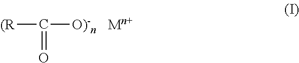

wherein R is a (C₇ to C₁₉) alkyl group, Mⁿ⁺ is a divalent cation, and n is the number 2, and
at least one propellant.

### (K):

A cosmetic agent for treating keratinic fibers, particularly human hair, comprising in a liquid alcoholic cosmetic carrier containing at least one alcohol having 2 to 6 carbon atoms and 1 to 3 hydroxyl groups—
2 to 20 wt. %, particularly 3 to 15.0 wt. %, of at least one at least one starch compound dispersed as solid particles, chosen from at least one—optionally modified—polycondensation product of D-glucose obtained from starch from potatoes, corn, rice, peas, acorns, chestnuts, barley, wheat, bananas, sago, millet, sorghum, oats, barley, rye, beans, sweet potato, maranta or cassava,
0.01 to 5.0 wt. %, particularly 0.05 to 1.0 wt. %, of at least one (C₈ to C₂₀) nnonocarboxylic acid compound of formula (I)

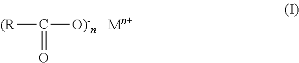

wherein R is a (C₇ to C₁₉) alkyl group, Mⁿ⁺ is a divalent cation, and n is the number 2, and
at least one propellant.

### (L):

A cosmetic agent for treating keratinic fibers, particularly human hair, comprising in a liquid alcoholic cosmetic carrier containing at least one alcohol having 2 to 6 carbon atoms and 1 to 3 hydroxyl groups—
2 to 20 wt. %, in particular from 3 to 15.0 wt. %, of at least one starch compound dispersed as solid particles, chosen from at least one—optionally modified—polycondensation product of D-glucose obtained from starch from potatoes, corn, rice, peas, acorns, chestnuts, barley, wheat, bananas, sago, millet, sorghum, oats, barley, rye, beans, sweet potato, maranta or cassava,
0.01 to 5.0 wt. %, particularly 0.05 to 1.0 wt. %, of at least one (C₈ to C₂₀) monocarboxylic acid compound chosen from ammonium stearate, calcium distearate, magnesium distearate, zinc distearate, ammonium isostearate, calcium diisostearate, magnesium diisostearate, zinc diisostearate, and
at least one propellant.

The particularly preferred embodiments (A) to (L) can be combined mutatis mutandis with the parameters of the features not mentioned there but referred to previously as being preferred.

Agents according to the invention in a preferred embodiment additionally contain at least one silicone, particularly at least one silicone oil.

Suitable silicone oils according to the invention include dialkyl and alkylaryl siloxanes, such as dimethyl polysiloxane and methyl phenyl polysiloxane, as well as their alkoxylated, quaternized or anionic derivatives. Preferred are cyclic and linear polydialkylsiloxanes, their alkoxylated and/or aminated derivatives, dihydroxy polydimethylsiloxanes and polyphenyl alkyl siloxanes.

The term “silicone oils” is understood by one skilled in the art as a number of structures of organosilicon compounds. Firstly, they are understood to mean the dimethiconols.

The following commercial products are mentioned as examples of these products: Botanisil NU-150M (Botanigenics), Dow Corning 1-1254 Fluid, Dow Corning 2-9023 Fluid, Dow Corning 2-9026 Fluid, Ultrapure Dimethiconol (Ultra Chemical), Unisil SF-R (Universal Preserve), X-21-5619 (Shin-Etsu Chemical Co.), Abil OSW 5 (Degussa Care Specialties), ACC DL-9430 Emulsion (Taylor Chemical Company), AEC Dimethiconol & Sodium Dodecylbenzenesulfonate (A & E Connock (Perfumery & Cosmetics) Ltd.), B C Dimethiconol Emulsion 95 (Basildon Chemical Company, Ltd.), Cosmetic Fluid 1401, Cosmetic Fluid 1403, Cosmetic Fluid 1501, Cosmetic Fluid 1401 DC (all the above Chemsil Silicones, Inc.), Dow Corning 1401 Fluid, Dow Corning 1403 Fluid, Dow Corning 1501 Fluid, Dow Corning 1784 HVF Emulsion, Dow Corning 9546 Silicone Elastomer Blend (all the above Dow Corning Corporation), Dub Gel SI 1400 (Stearinerie Dubois Fils), HVM 4852 Emulsion (Crompton Corporation), Jeesilc 6056 (Jeen International Corporation), Lubrasil, Lubrasil DS (both Guardian Laboratories), Nonychosine E, Nonychosine V (both Exsymol), SanSurf Petrolatum-25, Satin Finish (both Collaborative Laboratories, Inc.), Silatex-D30 (Cosmetic Ingredient Resources), Silsoft 148, Silsoft E-50, Silsoft E-623 (all the above Crompton Corporation), SM555, SM2725, SM2765, SM2785 (all the above GE Silicones), Taylor T-Sil CD-1, Taylor TME-4050E (all Taylor Chemical Company), TH V 148 (Crompton Corporation), Tixogel CYD-1429 (Sud-Chemie Performance Additives), Wacker-Belsil CM 1000, Wacker-Belsil CM 3092, Wacker-Belsil CM 5040, Wacker-Belsil DM 3096, Wacker-Belsil DM 3112 VP, Wacker-Belsil DM 8005 VP, Wacker-Belsil DM 60081 VP (all the above Wacker-Chemie GmbH).

Dimethicones form the second group of silicones which can be present in the agent according to the invention. These can be linear and branched, as well as cyclic or cyclic and branched.

Dimethicone copolyols form another group of suitable silicones. Appropriate dimethicone copolyols are commercially available and are marketed, for example, by Dow Corning with the name Dow Corning® 5330 Fluid.

The teaching of the invention also includes the fact that the dimethiconols, dimethicones and/or dimethicone copolymers can already be present as an emulsion. The corresponding emulsion of dimethiconols, dimethicones and/or dimethicone copolyols can be produced both after the production of the corresponding dimethiconols, dimethicones and/or dimethicone copolyols from these and by the conventional methods for emulsification known to the person skilled in the art. For this purpose, as auxiliary agents to produce the corresponding emulsions, both cationic, anionic, non-ionic or zwitterionic surfactants and emulsifiers can be used as auxiliary substances. The emulsions of the dimethiconols, dimethicones and/or dimethicone copolyols can also be produced directly by an emulsion polymerization process. Processes of this type are also well known to one skilled in the art.

If dimethiconols, dimethicones and/or dimethicone copolyols are used as an emulsion, the droplet size of the emulsified particles according to the invention is 0.01 to 10000 μm, preferably 0.01 to 100 μm, particularly preferably 0.01 to 20 μm and most particularly preferably 0.01 to 10 μm. The particle size is determined in this case by the light scattering method.

If branched dimethiconols, dimethicones and/or dimethicone copolyols are used, this means that the branching is greater than random branching, which occurs by chance through impurities of the respective monomers. Within the meaning of the present invention, therefore, branched dimethiconols, dimethicones and/or dimethicone copolyols are to be understood as having a degree of branching greater than 0.01%. A degree of branching greater than 0.1% is preferred, and most particularly preferably greater than 0.5%. The degree of branching here is determined from the ratio of unbranched monomers to branched monomers (i.e., the quantity of tri- and tetrafunctional siloxanes). According to the invention, low-branched and high-branched dimethiconols, dimethicones and/or dimethicone copolyols can be particularly preferred.

Most particularly preferably suitable silicones are selected from cyclic silicones. Of these, those of formula (Si-1) are particularly preferred,

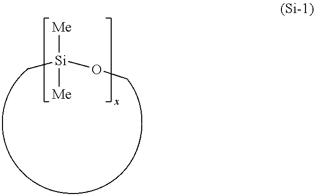

wherein x is a number from 4 to 10, preferably from 4 to 7 and particularly 4, 5 or 6. In particular, the agent of this embodiment most preferably contains octamethylcyclotetrasiloxane and/or decamethylcyclopentasiloxane.

Cyclic dimethicones are designated as Cyclomethicones according to INCI and are under the trade name Xiameter PMX 0244 Cyclotetrasiloxane, Xiameter PMX 0344 Cyclosiloxane Blend, Xiameter PMX 0245 Cyclopentasiloxane and Xiameter PMX 0345 Cyclosiloxane Blend from the manufacturer Xiameter.

Agents according to the invention contain silicones preferably in amounts of 0.01 wt. % to 15 wt. %, more preferably 0.05 to 2 wt. %, based on total agent.

In particular, conditioners should be mentioned as suitable auxiliary substances and additives.

As a conditioner, agents according to the invention optionally also contain at least one plant extract. These extracts are usually produced by extraction of the whole plant. In certain cases, however, it may also be preferred to produce the extracts exclusively from the flowers and/or leaves of the plant.

Suitable plant extracts are obtained by extraction with organic solvents (e.g., ethanol, isopropanol, diethyl ether, naphtha, benzene, chloroform) or by steam distillation. According to the invention, the extracts of bamboo, linseed, water lily, green tea, oak bark, stinging nettle, witch hazel, hops, henna, chamomile, burdock root, horsetail, whitethorn, lime blossom, almond, aloe vera, spruce, horse chestnut, sandalwood, juniper, coconut, mango, apricot, lime, wheat, kiwi, melon, orange, grapefruit, sage, rosemary, birch, mallow, cuckoo flower, wild thyme, yarrow, thyme, melissa, rest harrow, coltsfoot, marsh mallow, meristem, ginseng and ginger root are especially preferred.

The additional plant extract is preferably present in the agent in an amount of 0.05 wt. % to 1.0 wt. %, particularly 0.1 wt. % to 0.5 wt. %, based on weight of the cosmetic agent.

As a conditioner it is possible to use a cationic surfactant. Cationic surfactants of the type of the quaternary ammonium compounds, the ester quats and the amidoamines are preferred in this case. Preferred quaternary ammonium compounds are ammonium halides, particularly chlorides and bromides, such as alkyltrimethylammonium chlorides, dialkyl-dimethylammonium chlorides and trialkylmethylammonium chlorides (e.g., cetyltrimethylammonium chloride, stearyltrimethylammonium chloride, distearyldimethylammonium chloride, lauryldimethylammonium chloride, lauryldimethylbenzylammonium chloride and tricetylmethylammonium chloride), and the imidazolium compounds know by the INCI names Quaternium-27 and Quaternium-83. The long alkyl chains of the above-mentioned surfactants preferably have 10 to 18 carbon atoms. Since the addition of surface-active substances can have a negative effect on the hydrophobic properties of the hydrophobized silicon dioxide and thus on the stability of the cosmetic agent according to the invention, the amount of conditioning surfactant should be carefully adapted to the overall composition. The addition of surfactant components is preferably omitted.

Furthermore, cationized protein hydrolyzates are included in the cationic polymers, with the underlying protein hydrolyzate being of animal origin (e.g., from collagen, milk or keratin), of plant origin (e.g., from wheat, corn, rice, potatoes, soybean or almonds), from marine life forms (e.g., from fish collagen or algae), or protein hydrolyzates obtained by biotechnology. The protein hydrolyzates on which the cationic derivatives according to the invention are based can be obtained from the corresponding proteins by chemical, particularly alkaline or acidic hydrolysis, by enzymatic hydrolysis and/or a combination of the two types of hydrolysis. The hydrolysis of proteins generally produces a protein hydrolyzate with a molecular weight distribution of about 100 Daltons up to several thousand Daltons. Preferred are those cationic protein hydrolyzates of which the underlying protein section has an average molecular weight (weight average) of 100 up to 25000 Daltons, preferably 250 to 5000 Daltons. Furthermore, cationic protein hydrolyzates include quaternized amino acids and mixtures thereof. Quaternizing of the protein hydrolyzates or the amino acids is often performed using quaternary ammonium salts, such as N,N-dimethyl-N-(n-alkyl)-N-(2-hydroxy-3-chloro-n-propyl)ammonium halides. Furthermore, cationic protein hydrolyzates can also be further derivatized. Commercially available products mentioned under their INCI names in the “International Cosmetic Ingredient Dictionary and Handbook”, (7th Ed. (1997), The Cosmetic, Toiletry, and Fragrance Association 1101 17th Street, N.W., Suite 300, Washington, D.C. 20036-4702) may be mentioned as typical examples of cationic protein hydrolyzates and derivatives according to the invention.

Plant-based cationic protein hydrolyzates and derivatives are preferred.

As a conditioner, it is also possible to use at least one vitamin, provitamin, vitamin precursor and/or derivative thereof.

According to the invention, those vitamins, pro-vitamins and vitamin precursors which are generally assigned to the groups A, B, C, E, F and H are preferred. Particularly preferred are vitamins belonging to the B group or to the vitamin B complex, most particularly preferably vitamin B₅ (pantothenic acid, panthenol and pantolactone).

Other suitable conditioners are protein hydrolyzates and/or derivatives thereof, the use of protein hydrolyzates of plant origin (e.g., soybean, almond, pea, potato and wheat protein hydrolyzates) being preferred. These products are obtainable, for example, under the trade names Gluadin® (Cognis), DiaMin® (Diamalt), Lexein® (Inolex), Hydrosoy® (Croda), Hydrolupin® (Croda), Hydrosesame® (Croda), Hydrotritium® (Croda) and Crotein® (Croda).

Although the use of protein hydrolyzates as such is preferred, mixtures of amino acids obtained otherwise can optionally be used instead. It is also possible to use derivatives of protein hydrolyzates, for example, in the form of their fatty acid condensation products. These products are marketed, for example, under the names Lamepon® (Cognis), Lexein® (Inolex), Crolastin® (Croda), Crosilk® (Croda) or Crotein® (Croda).

The teaching of the invention includes all isomeric forms, such as cis/trans isomers, diastereomers and chiral isomers.

It is also possible according to the invention to use a mixture of several protein hydrolyzates.

In addition to the conditioners, other auxiliary substances and additives can also be added.

By adding a UV filter, both the preparations themselves and the fibers being treated can be protected from harmful effects of UV radiation. It can therefore be advantageous to also add at least one UV filter to the cosmetic agents. Suitable UV filters are not subject to any general restrictions in terms of their structure and their physical properties. Rather, all UV filters are suitable which can be used in the cosmetics sector and have an absorption maximum in the UVA (315-400 nm), UVB (280-315 nm) or UVC (<280 nm) range. UV filters with an absorption maximum in the UVB range, particularly in the range from about 280 to about 300 nm, are particularly preferred.

Preferred UV filters according to the invention can be chosen, for example, from substituted benzophenones, p-aminobenzoic acid esters, diphenylacrylic acid esters, cinnamic acid esters, salicylic acid esters, benzimidazoles and o-aminobenzoic acid esters. 2-Hydroxy-4-methoxybenzophenone-5-sulfonic acid and its sodium salt (Benzophenone-4; Uvinul®MS 40; Uvasorb®S 5) may be mentioned here by way of example.

In a particular embodiment, cosmetic agents according to the invention also contain one or more substantive dyes. This makes it possible for the keratinic fibers being treated not only to be temporarily structured when the agent is applied, but at the same time also dyed. This may be desirable particularly if only temporary dyeing (e.g., with striking fashion colors) is desired, which can be removed from the keratinic fibers again simply by washing.

Furthermore, the cosmetic agents can contain alkalizing agents, generally alkali or alkaline earth hydroxides, ammonia or organic amines. Preferred alkalizing agents are monoethanolamine, monoisopropanolamine, 2-amino-2-methylpropanol, 2-amino-2-methyl-1,3-propanediol, 2-amino-2-ethyl-1,3-propanediol, 2-amino-2-methylbutanol and triethanolamine as well as alkali and alkaline earth metal hydroxides. Monoethanolamine, triethanolamine and 2-amino-2-methylpropanol and 2-amino-2-methyl-1,3-propanediol are particularly preferred in the context of this group. Use of co-amino acids such as co-aminocaproic acid as alkalizing agents is also possible.

In the above-mentioned embodiments, the features of the agent according to the invention referred to as preferred, particularly the quantities used, each apply mutatis mutandis.

If the agents according to the invention are in the form of foam, they are discharged from a dispensing device that is suitable for foaming, which is either a pressurized gas container additionally filled with a propellant (“aerosol container”) or a non-aerosol container. In this case, the disperse system of the cosmetic carrier is present as foam.

The present invention secondly provides an aerosol dispensing container comprising at least one valve, at least one nozzle and at least one storage container containing a cosmetic agent containing in a cosmetic carrier at least one starch compound, at least 0.01 wt. % of a (C₈ to C₂₀) monocarboxylic acid compound, and at least one propellant.

Pressurized gas containers from which a product is dispensed through a valve due to the internal gas pressure of the container are referred to as “aerosol dispensing containers”. Conversely, a container under standard pressure from which a product is dispensed by mechanical action using a pump system is defined as a “non-aerosol dispensing container”. The cosmetic agent of the invention can be dispensed either as a spray or as foam from the aerosol dispensing container.

Particularly good effects according to the invention are achieved if the internal pressure of the aerosol dispensing container (particularly the storage container) is at least 1.8 bar, and particularly at least 2.5 bar.

In this case, the cosmetic agents described in the first subject matter of the invention, particularly the preferred embodiments thereof, are preferably suitable in the aerosol dispensing container.

The present invention thirdly provides a method of treating keratinic fibers, particularly human hair, wherein a cosmetic agent of the first subject matter of the invention is applied onto the keratinic fibers as an aerosol.

It is preferred if an aerosol dispensing container of the second subject matter of the invention is used to apply the cosmetic agent.

The following examples are intended to explain the subject matter of the present invention without limiting it in any way.

### EXAMPLES

The following formulations were prepared:

| Raw material | E1 [wt. %] | E2 [wt. %] | E3 [wt. %] |
|---|---|---|---|
| Rice starch | 10.0 | 10.0 | — |
| (Remy ® UFG KA, Erbslöh) | | | |
| Aluminum starch | — | — | 10.0 |
| octenylsuccinate | | | |
| (Dry Flo Plus) | | | |
| Zinc distearate | 0.2 | — | 0.2 |
| Magnesium distearate | — | 0.2 | — |
| Perfume | 0.2 | 0.2 | 0.2 |
| Ethanol | 14.6 | 14.6 | 14.6 |
| Isobutane | 75.0 | 75.0 | 75.0 |

All of the components of the respective agent, apart from the propellant (isobutane), were thoroughly mixed with stirring and the resulting dispersion of particulate rice starch was charged into a storage container of an aerosol dispensing container. The storage container was sealed with a mounting cup encompassing a CA39F valve from Lindal. The propellant was then charged into the aerosol storage container through the valve and finally an actuator with an ST34013.3 nozzle from Lindal was attached. In this way, an aerosol dispensing container each having an internal pressure of 2.2 bar was prepared for each of agents E1, E2 and E3.

The respective aerosol dispensing containers could be emptied as a spray without the valve or nozzle becoming excessively clogged or even blocked.

We claim:

1. A cosmetic agent for treating keratinic fibers comprising in a cosmetic carrier:
at least one starch compound,
at least 0.01 wt. % of a (C₈ to C₂₀) monocarboxylic acid compound of formula (I) 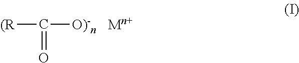
wherein R is a (C₇ to C₁₉) alkyl group, Mⁿ⁺ is an n-valent cation and n is 1, 2 or 3, and at least one propellant.

2. The cosmetic agent according to claim 1, wherein the at least one starch compound is present as solid particles dispersed in the cosmetic carrier.

3. The cosmetic agent according to claim 1, wherein the at least one starch compound is chosen from at least one—optionally modified—polycondensation product of D-glucose obtained from starch from potatoes, corn, rice, peas, acorns, chestnuts, barley, wheat, bananas, sago, millet, sorghum, oats, barley, rye, beans, sweet potato, maranta or cassava.

4. The cosmetic agent according to claim 1, wherein the at least one starch compound is present in an amount of 2 to 20 wt. %, based on total weight of the agent.

5. The cosmetic agent according to claim 1, wherein Mⁿ⁺ in formula (I) is chosen from metal cations of physiologically acceptable metals of groups Ia, Ib, IIa, IIb, IIIb, VIa or VIII of the periodic table, ammonium ions, and cationic organic compounds with a quaternized nitrogen atom.

6. The cosmetic agent according to claim 1, wherein R in formula (I) is a linear or branched (C₇ to C₂₀) alkyl group.

7. The cosmetic agent according to claim 6, wherein R in formula (I) is n-heptyl, n-nonyl, n-undecyl, n-tridecyl, n-pentadecyl or n-heptadecyl.

8. The cosmetic agent according to claim 1, wherein the (C₈ to C₂₀) monocarboxylic acid compounds of formula (I) are present in an amount of 0.01 to 5.0 wt. %, based on total weight of the agent.

9. The cosmetic agent according claim 1 further comprising at least one silicone.

10. An aerosol dispensing container comprising at least one valve, at least one nozzle and at least one storage container comprising a cosmetic agent according to claim 1.

11. The aerosol dispensing container according to claim 9, wherein the internal pressure of the aerosol dispensing container is at least 1.8 bar.

12. A method of treating keratinic fibers comprising applying a cosmetic agent according to claim 1 onto the keratinic fibers in the form of an aerosol.

13. The method according to claim 11, wherein the aerosol is contained in an aerosol dispensing container comprising at least one valve, at least one nozzle and at least one storage container comprising a cosmetic agent according to claim 1.
